(19) United States
(12) Patent Application Publication
(10) Pub. No.: US 2023/0368118 A1
(43) Pub. Date: Nov. 16, 2023

(54) System and Method for Predictive Analytics

(71) Applicant: Northeastern University, Boston, MA (US)

(72) Inventors: Cordula A. Robinson, Boxborough, MA (US); Nicholas Beauchamp, Belmont, MA (US); Nan Gao, Boston, MA (US)

(21) Appl. No.: 18/315,190

(22) Filed: May 10, 2023

Related U.S. Application Data
(60) Provisional application No. 63/364,482, filed on May 10, 2022.

(51) Int. Cl. G06Q 10/067

(52) U.S. Cl. CPC G06Q 10/067; G06F 40/205

(57) ABSTRACT

A computer-implemented method and corresponding system perform predictive analytics. The system comprises a machine learning (ML) predictor model and diverse ML models coupled to the ML predictor model. The ML predictor combines multiple independent data streams received from respective diverse ML models of the diverse ML models. The multiple independent data streams include respective features indicative of an observable event. The combining produces at least one combination of the respective features. The ML predictor generates a prediction of the observable event. The prediction is based on the at least one combination produced. The ML predictor outputs a representation of the prediction generated. The combining enables temporally and geographically consistent insights, derived via machine learning, to be employed to generate a more robust prediction relative to a prediction generated using conventional techniques.

### RELATED APPLICATION

This application claims the benefit of U.S. Provisional Application No. 63/364,482, filed on May 10, 2022. The entire teachings of the above application are incorporated herein by reference.

### GOVERNMENT SUPPORT

This invention was made with government support under Intelligence Advance Research Projects Activity (IARPA) Contract Nos.: 2021-21051400001 and 2021-21030200007 from the Office of Director of National Intelligence, IARPA. The government has certain rights in the invention.

### BACKGROUND

Predictive analytics may include the use of data, statistical methods, and machine learning techniques to identify the likelihood of future outcomes based on historical data. A goal of predictive analytics is to go beyond knowing what has happened to providing a best assessment of what will happen in the future. Many companies use predictive models to forecast inventory and manage resources. Airlines use predictive analytics to set ticket prices. Hotels try to predict the number of guests for any given night to maximize occupancy and increase revenue. As such, predictive analytics enables organizations to function more efficiently.

### SUMMARY

According to an example embodiment, a computer-implemented method for predictive analytics comprises combining, by a machine-learning (ML) predictor model, multiple independent data streams received from respective diverse ML models. The multiple independent data streams include respective features indicative of an observable event. The combining produces at least one combination of the respective features. The computer-implemented method further comprises generating, by the ML predictor model, a prediction of the observable event. The prediction is based on the at least one combination produced. The computer-implemented method further comprises outputting, by the ML predictor model, a representation of the prediction generated.

The observable event may include at least one of: an outbreak of an infectious disease, release of a chemical or biological warfare agent, pattern of migration, or other observable event.

The representation may be a visual representation. Outputting of the representation may include outputting the visual representation to an electronic display device and displaying the visual representation on the electronic display device.

Combining the multiple independent data streams may include ranking the respective features and weighting the respective features ranked.

The computer-implemented method may further comprise producing the multiple independent data streams. Producing the at least one combination may include performing, by at least one respective diverse ML model of the respective multiple diverse ML models, natural language processing (NLP) on input data. The NLP may include extracting natural language information from the input data. At least a portion of the respective features may include the natural language information extracted.

The computer-implemented method may further comprise customizing a respective diverse ML model of the respective diverse ML models to produce an independent data stream of the multiple independent data streams based on social media analytics, mobility data analytics, dynamic data analytics, or static data analytics.

The computer-implemented method may further comprise customizing the respective multiple diverse ML models to produce the respective features by filtering input data based on a database of terms. The terms may include natural language. The terms may be categorized into sentiments in the database.

The computer-implemented method may further comprise producing the multiple independent data streams by the respective multiple diverse ML models. The multiple independent data streams produced may be associated with a common geographical area, common timeframe, and common user pool. At least a portion of users of the common user pool may be determined to be located within the common geographical area for at least a portion of the common timeframe.

The computer-implemented method may further comprise producing the multiple independent data streams, by the multiple diverse ML models, with a consistent format. The consistent format may be associated with NLP data.

Generating the prediction may include employing at least one of: statistical modeling, Bayesian modeling, least absolute shrinkage, and selection operator (Lasso) regression modeling, or topic modeling.

According to another example embodiment, a system for predictive analytics comprises a machine learning (ML) predictor model and diverse ML models coupled to the ML predictor model. The ML predictor model is configured to combine multiple independent data streams received from respective diverse ML models of the diverse ML models. The multiple independent data streams include respective features indicative of an observable event. The combining produces at least one combination of the respective features. The system is further configured to generate a prediction of the observable event. The prediction is based on the at least one combination produced. The system is further configured to output a representation of the prediction generated.

Alternative system embodiments parallel those disclosed above in connection with the example computer-implemented method embodiment.

According to yet another example embodiment, a non-transitory computer-readable medium for predictive analytics has encoded thereon a sequence of instructions which, when loaded and executed by at least one processor, causes the at least one processor to combine multiple independent data streams received from respective diverse machine learning (ML) models. The multiple independent data streams include respective features indicative of an observable event. The combining produces at least one combination of the respective features. The system is further configured to generate a prediction of the observable event. The prediction is based on the at least one combination produced. The system is further configured to output a representation of the prediction generated.

Alternative non-transitory computer-readable medium embodiments parallel those disclosed above in connection with the example computer-implemented method embodiment.

It should be understood that example embodiments disclosed herein can be implemented in the form of a method, apparatus, system, or non-transitory computer readable medium with program codes embodied thereon.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is a block diagram of an example embodiment of a computing environment for predictive analytics.

FIG. 2 is a block diagram of an example embodiment of a system for predictive analytics.

FIG. 3 is a flow diagram of an example embodiment of a computer-implemented method for predictive analytics.

FIG. 4 is a flow diagram of an example embodiment of a method for large-scale geolocation of non-geolocated tweets.

FIG. 5 is a block diagram of an example of the internal structure of a computer in which various embodiments of the present disclosure may be implemented.

### DETAILED DESCRIPTION

A description of example embodiments follows.

An example embodiment disclosed herein includes an anticipatory mapping tool which may include a machine learning (ML) predictor model, disclosed further below. The ML predictor model may also be referred to interchangeably herein as a geospatial-temporal modeling and mapping (G-MAP) predictor. Such a model may be used to predict a next viral hot spot, in the case of an epidemic or pandemic, or to predict a different geo-spatiotemporal event for non-limiting examples. An example embodiment enables an intelligence community, emergency, public health, and other relevant authorities to have accurate insights into potential disease outbreaks via access to data that does not result in undermining public trust or support owing to protection of Personally Identifiable Information (PII).

Example methods developed herein “listen” to insights from those communities with, for example, rising COVID-19 rates to create empirical training data for Natural Language Processing (NLP). By creating a training data set of high accuracy, reliable Artificial Intelligence (AI) models can be built with powerful prediction performances. NLP provides means to forecast outbreaks in near real-time with extensibility to future pandemics, for non-limiting example. Researchers may leverage multi-geospatial-temporal data streams, along with dynamic topic models populated with keywords of symptomatic language, behavioral responses, and comments about pre-infection risk behaviors. Insights for NLP training may be derived from diverse neighborhoods over an input time period (date range). Through retroactive identification of areas of emergence, geographic sentiment analysis may be applied to define taxonomies of social media discourse for seeding a scalable, real-time AI model based on NLP. The use of empirical data to inform taxonomy definition is novel from other known techniques.

It is useful to provide rapid capability to respond to dynamic shifts in, for non-limiting example, a pandemic, such as COVID-19 or other pandemic, as a means to forecast outbreaks in near real-time, with extensibility to future virus outbreaks or pandemics, among other possible uses. Using NLP, an example embodiment disclosed herein provides for in-place and traveling personnel to be able to retrieve and/or receive local enhanced warning pandemic alerts—as much as every 10 minutes for non-limiting example—for situational awareness and risk appraisal, while not exposing sensitive data.

An example embodiment may monitor a social media (e.g., Twitter® for non-limiting example) pipeline, which enables an example embodiment to “listen” to insights from communities with rising COVID-19 rates, keywords, and dynamic topic models, which can incorporate behavioral responses and pre-infection risk behaviors, as well as symptomatic language for non-limiting example. For convenience, example embodiments disclosed herein may be in the context of Twitter; however, it should be understood that any social media platform that supports similar functionality as employed by embodiments presented herein or equivalents thereof may alternatively be employed. Also, while example embodiments disclosed herein may be described in reference to COVID-19 as an example pandemic, it should be understood that other diseases or non-disease applications are contemplated to be within the scope of embodiments disclosed herein or equivalents thereof.

An example embodiment enables geographic content and sentiment analysis of content derived through social media, such as Twitter. Twitter changed its location sharing policy, meaning the number of geo-located tweets is greatly limited. An example embodiment enables access to large-scale, localized data, applying a user timeline method. For non-limiting example, Twitter user identifiers (ids) may be collected for geographic areas of interest, such as within a 3-mile radius of the given latitude/longitude and no older than one week for non-limiting example. A timeline of tweets of such users may be retrieved. Thus, an example embodiment may “listen” to those communities with rising COVID-19 rates as a preparatory step to creating NLP-generated topic models that are calibrated to known outbreaks. An example embodiment may employ a new way to source social media Twitter data and retain a focus on the geography and time of an event.

Currently, analytical models rely on data produced after an outbreak occurs compared with an example embodiment of an approach described herein that enables reliable predictions to potentially fall within the incubation period, pre-medical diagnosis. Additionally, models described herein may be derived locally through thorough engagement with a rich-content form of social media that gives fine-grained geographical variation and insight into opinion and behavior, which is usually missing in existing analytical approaches that tend to begin at a country or state level, etc. Thus, many issues, including but not limited to crises, like a pandemic, are described in terms of broad societal consequences; however, they unfold locally, with hotspots and their impacts starting in individual towns and neighborhoods. An example embodiment disclosed herein supports such granularity, and this is distinguishing from known systems and provides a unique advantage.

An example embodiment disclosed herein may include implementing a plurality of tasks, a synopsis of which may include, for non-limiting example: 1) Neighborhood-level Analysis: Apply empirically derived data from an area cohort to characterize dynamic information of interest (e.g., virus health). Utilize such derived data together with data form traditional data sources (e.g., for different age groups, styles of living and housing, race, education levels, income levels) aggregated to neighborhoods to detect social structures that may influence dynamic a variable (e.g., COVID-19's spread). Proxies may be established, e.g., through regression. 2) Mobility Analysis (optional): Conduct mobility analysis to predict population mobility as it pertains to impact on dynamic information being studied, e.g., diffusion and direction of transmission of virus to predict infection and virus spread. 3) Geographic Content: Perform predictive modeling based on 1) and 2), disclosed above, add geographic content and sentiment analysis derived through social media to a) identify words, topics, and dynamic changes in discourse and communications that are associated with dynamic phenomena (e.g., COVID-19 outbreaks) and to b) “listen” to those communities. 4) ML/AI NLP: Test social media natural language information derived in 3) with geographic and dynamic (disease) measures to dynamically detect textual correlates (e.g., of disease hotspots to identify emerging hotspots prior to medical measurements). 5) Apply privacy protection: Data can be sensitive in nature and include personally identifiable information such as internet protocol (IP) addresses. Such a task may anonymize data, for example, by aggregating at a block level, and may aggregate or anonymize via parsing analysis so an individual user cannot be tracked. An example embodiment may substitute IP addresses with pseudonymous identifiers and follow standard ethical practices from the fields of data science and computer networks.

An example embodiment disclosed herein may employ novel use of empirical data for NLP taxonomy definition. Insights may be derived using empirical geospatial and mobility data from the diverse neighborhoods. Areas of emergence may be the focus of geographic sentiment analysis to define taxonomies of social media discourse for seeding a scalable AI that is based on NLP.

An example embodiment disclosed herein may employ a creative coalition of techniques that are novel. For non-limiting example, such techniques may include: 1) neighborhood-level analysis for a city or other geographical location with dynamic data and social structure and establish proxies; 2) mobility analysis to locate hotspots of infection; 3) geographic content from social media with best practices to derive information; 4) ML/AI NLP-Test social media natural language information with geographic and dynamic measures to continuously detect textual correlates for real-time updated models that support interactive spatio-temporal queries; and 5) integrating 1) through 4), disclosed above, with anonymization.

An example embodiment disclosed herein may include a creation of a substratum tracking and listening engine with common operating picture for understanding subtle cues. An example embodiment may include techniques from NLP to model subtle cues and extract message signals from social media data. An example embodiment may encompasses use of modern digital data via linking spatial data from various sources at multiple geographic scales.

An example embodiment may employ aggregation and anonymization of data, for non-limiting example, by substituting IP addresses with pseudonymous identifiers. Unlike traditional contact tracing approaches, an example embodiment disclosed herein is not uniquely dependent on cellphone data, and/or 5G technology and may be deployed on a cloud platform.

An example embodiment disclosed herein may retrieve geolocated open source intelligence (OSINT) data using an example embodiment of a pipeline. The pipeline may be used to retrieve data from social media platforms (e.g., starting with Twitter) in a way that enables locations of posts to be known. The example embodiment may include a layer that ensures anonymization, so no personally identifiable information is collected. This could be useful in many applications as it is a means to create a substratum tracking and listening engine for subtle cues without profiling.

An example embodiment disclosed herein may be employed for a number of use cases and is not limited to being employed to a use case(s) disclosed herein. For non-limiting example, an example embodiment disclosed herein may be employed for predicting a next COVID-19 hotspot and may enable the intelligence community, emergency, public health and other relevant authorities to have accurate insights into potential disease outbreaks via access to data that does not result in undermining public trust. An example embodiment may employ subtle cues to quickly and accurately predict the spread and impact of a) infectious diseases b) chemical/biological warfare agents. An example embodiment disclosed herein may be employed in an OSINT software service for new Digital Exploitation Techniques to understand patterns of life or may be used for other predictive analytics.

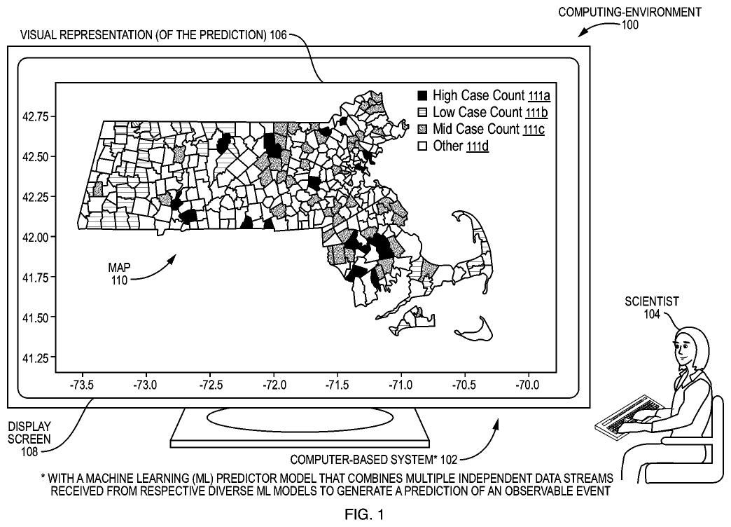

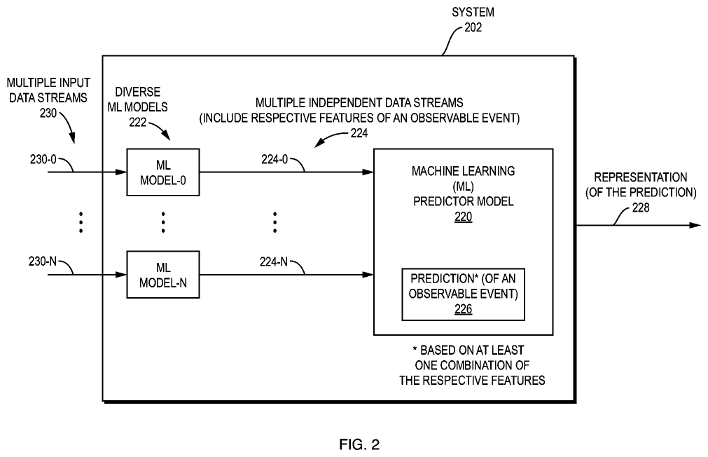

FIG. 1 is a block diagram of an example embodiment of a computing environment 100 for predictive analytics. The computing environment 100 includes a computer-based system 102. A scientist 104 is viewing a visual representation 106 displayed on a display screen 108 of the computer-based system 102. For non-limiting example, the visual representation 106 is a map 110 that indicates predictions of case counts of an infectious disease within different regions of the map 110. Such regions are indicated on the map 110 as having predictions of a high case count 111a, low case count 111b, mid case count 111c, or other case count 111d for the infectious disease for non-limiting example. In the example embodiment, the computer-based system 102 comprises a machine learning (ML) predictor model (not shown) and diverse ML models (not shown) coupled to the ML predictor model for generating the predictions, such as disclosed below with regard to FIG. 2.

FIG. 2 is a block diagram of an example embodiment of a system 202 for predictive analytics. The system 202 may be employed by the computer-based system 102 of FIG. 1, disclosed above. Continuing with reference to FIG. 2, the system 202 comprises a machine learning (ML) predictor model 220 and diverse ML models 222 (e.g., ML model-0, . . . , ML model-N) coupled to the ML predictor model 220. The ML predictor model 220 is configured to combine multiple independent data streams 224 (e.g., 224-0, . . . , 224-N) received from respective diverse ML models of the diverse ML models 222. The multiple independent data streams 224 include respective features (not shown) indicative of an observable event (not shown). The combining produces at least one combination (not shown) of the respective features. The system is further configured to generate a prediction 226 of the observable event. The prediction 226 is based on the at least one combination produced. The system 202 is further configured to output a representation 228 of the prediction 226 generated.

The observable event may include at least one of: an outbreak of an infectious disease, release of a chemical or biological warfare agent, pattern of migration, or other observable event, for non-limiting examples.

The multiple independent data streams 224 may be temporally and geographically consistent. Such data streams may include features derived from machine learning performed by the diverse ML models 222. Such features may represent insights/predictions that are further refined by the ML predictor model 220 to generate the prediction 226.

The system 202 may be a computer-based system, such as the computer-based system 102 of FIG. 1, disclosed above for non-limiting example. With reference to FIG. 1 and FIG. 2, the computer-based system (102, 202) may include an electronic display device, such as the display screen 108 for non-limiting example. The representation 228 may be a visual representation, such as the visual representation 106 for non-limiting example. The ML predictor model 220 may be further configured to output the visual representation 106 to the electronic display device (e.g., display screen 108). The electronic display device may be configured to display the visual representation 106.

Continuing with reference to FIG. 2, the ML predictor model 220 may be further configured to rank the respective features and weight the respective features ranked. At least one respective diverse ML model of the respective multiple diverse ML models 222 may be configured to produce an independent data stream of the multiple independent data streams 224 by performing natural language processing (NLP) on input data (not shown) that may included in a given input data stream (230-0, . . . , or 230-N) of multiple input data streams 230. Such multiple input data streams 250 may be independent high-dimensional data streams input to the diverse ML models 222. The NLP may include extracting natural language information (not shown) from the input data. At least a portion of the respective features may include the natural language information extracted.

A respective diverse ML model (e.g., ML model-0, . . . , ML model-N) of the respective diverse ML models 222 may be customized to produce an independent data stream (e.g., 224-0, . . . , 224-N) of the multiple independent data streams 224 based on social media analytics (not shown), mobility data analytics (not shown), dynamic data analytics (not shown), or static data analytics (not shown), for non-limiting examples. The respective multiple diverse ML models 222 may be customized to produce the respective features by filtering input data based on a database (not shown) of terms (not shown). The terms may include natural language. The terms may be categorized into sentiments in the database. The system 202 may be coupled to the database.

The multiple independent data streams 224 may be associated with a common geographical area, common timeframe, and common user pool for non-limiting example. At least a portion of users of the common user pool may be determined to be located within the common geographical area for at least a portion of the common timeframe. Such determining may be performed by the ML predictor model 220 based on at least one independent data stream of the multiple independent data streams 224.

The multiple independent data streams 224 may have a consistent format. The consistent format may be associated with NLP data (not shown). To generate the prediction 226, the ML predictor model 220 may be further configured to employ at least one of: statistical modeling, Bayesian modeling, least absolute shrinkage, and selection operator (Lasso) regression modeling, or topic modeling for non-limiting examples.

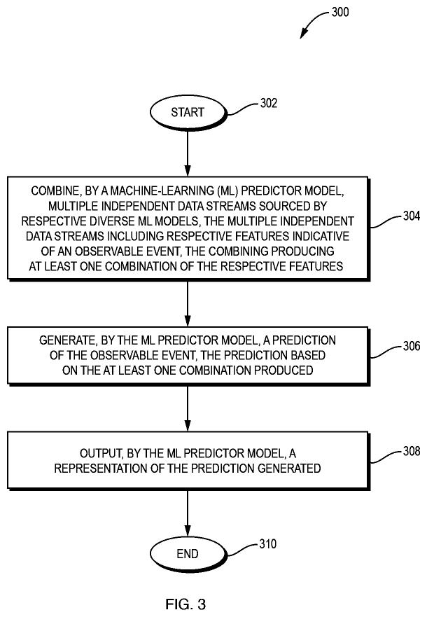

FIG. 3 is a flow diagram of an example embodiment of a computer-implemented method for predictive analytics (300). The method begins (302) and comprises combining, by a machine-learning (ML) predictor model, multiple independent data streams received from respective diverse ML models (304). The multiple independent data streams include respective features indicative of an observable event. The combining produces at least one combination of the respective features. The computer-implemented method further comprises generating, by the ML predictor model, a prediction of the observable event (306). The prediction is based on the at least one combination produced. The computer-implemented method further comprises outputting, by the ML predictor model, a representation of the prediction generated (308). The computer-implemented method thereafter ends (310) in the example embodiment. Further example embodiments are disclosed below with regard to non-limiting examples of uses cases.

Throughout the COVID-19 pandemic, there was a strong need for disease prediction. Existing predictive models often perform poorly, particularly during times of rapid case count change. These models routinely focus on curve shape and predict at a state or country level. According to an example embodiment, a virus may be examined as it unfolds locally, first at the census tract level for a city, then at a town level. Multiple independent data streams may be employed, such as, for non-limiting examples: 1) virus health data; 2) sociodemographic data; 3) municipality data; 4) mobility data; and 5) social media data. An example embodiment of a system disclosed herein may harness these immense data sources to improve real-world prediction, including understanding social and behavioral influences that may moderate and impact, for non-limiting example, COVID-19 case counts.

At a census tract level, an example embodiment may leverage a diverse array of real-time administrative and social media data. Such data may be employed to distinguish between relevant factors, or “proxies,” that either accelerate transmission, act as protective factors, or relate to underlying vulnerabilities in the population. At a town level, a first prediction may be performed that pertains to virus health using an Ordinary Least Squares (OLS) machine learning model. Creating such a regression model to predict a total number of COVID-19 cases per 1000 people in any town during any week of a pandemic is not trivial given the number of discrete variables in the demographic makeup of each town. It is useful to perform careful testing of explanatory sociodemographic variables, geographic weights, and lag importance to capture peaks and cyclicity that exist in extended case count and vaccine data. Including town-level mobility measures can further improve the predictive power and may account for unexplained case variation. This is especially true during a first phase of a pandemic, when behavioral responses are large, while diminishing later because of human learning and vaccinations, etc. Social media data are highly dense in time and space and span almost any conceivable topic of human endeavor. These data have potential to provide insights into the opinions and behaviors that underly predictions and may be used to generate new insights into Covid-related opinion.

Many issues, including but not limited to crises like a pandemic, are discussed in terms of broad societal consequences at state, county, national or even international levels. Viruses, however, unfold locally, starting in individual towns and neighborhoods. An example embodiment of a system disclosed herein anticipates (or “futurecasts”) case rates at a census tract and town level. At a census tract level, an example embodiment may use proxies drawn from various real-time administrative and social media data sets, and at the town level may employ social structure, social media, and mobility data. An example embodiment may create models that are more effective than using previous week case rates alone and may include proxies in the models to be interpretable in a way that might guide interventions, and retrieve new insights into COVID-19-associated behaviors and speech.

For non-limiting example, at a census tract level, a series of proxies may be employed that include both potential transmission events and protective behaviors from five data sets: 911 dispatches, 311 requests for government services, Craigslist postings, approved building permits, and cell phone-generated mobility records. Such proxies may be used to predict shifts in infection rates in future weeks over and above previous case rates, using fixed effects models. An example embodiment may leverage a Lasso technique to select the most meaningful proxies and their lags during a given wave of a pandemic. The fixed effects-lasso approach may be elaborated to weekly models, thereby quantifying how the importance of proxies shift across time. An example embodiment may create “futurecasts” of infection rates in each census tract for each week using parameters from a previous weeks' results, including an assessment of the accuracy of this approach.

At a town level, an example embodiment may employ aggregated Social Structure Data and virus health data. An example embodiment may employ a machine learning ordinary least squares (OLS) regression model, which learns from correlated and impactful engineered features. Such a model may be used to identify neighborhoods with escalating COVID-19 infection, also including an assessment of the accuracy and performance measures. An example embodiment may acquire and automate an acquisition process for dynamic data, such as COVID-19 case count, vaccine data, and Twitter data, to productionize predictive models for insertion into a Web App. An example embodiment of the Web App may be coupled to a live, relational database, where all datasets are scrubbed, and privacy protection is ensured. Such a Web App may be interactive.

For mobility data, an example embodiment may employ empirical modeling using Safegraph and Cuebiq data and theoretical modeling that uses Agent-Based Modeling (a computational model for simulating actions and interactions). Mobility data may be used to build a town-level mobility network (how many devices travel between two given towns on a given day), capture a level of risky contact frequency, and cluster different geographic regions according to behavioral response. Agent-Based Modeling and empirical results were found to be in close agreement, validating the level of importance of mobility parameters used and confirming that behavioral response plays a crucial role in the differential significance of local versus holistic network parameters.

For Natural Language Processing (NLP), an example embodiment may employ a new, open, public-API technique for geolocating Twitter data at a scale an order of magnitude denser than existing public methods. Such methods were developed and applied at a town level and generalizable to subnational regions worldwide. An example embodiment may build upon existing machine learning methods to construct a model that takes in tens of thousands of words and phrases at the town-week level and predicts COVID-19 cases one week into the future. An example embodiment of a text approach was found to improve upon a text-free baseline, particularly during times of rapid COVID-19 change. It is roughly as accurate 4 weeks in advance as the baseline model is 1 week in advance. An example embodiment may employ a new Bayesian model that, in combination with pre-existing Bayesian topic models, enables terms to be clustered and enables a model to not only identify how to predict COVID-19 cases, but also track how those effects change over time across different waves and pandemic periods.

At a town level, exploratory data analysis (EDA) provides a first means to evaluate high, medium, and low risk communities in a city and town. This approach provides the context in which to evaluate OLS model prediction accuracies for towns exhibiting vastly different COVID-19 case counts per 1000 people. The OLS model successfully predicts for all towns with all levels of risk and feature training provides insights into significant governing factors impacting COVID-19 case count rates. These are sociodemographic features; inter-municipality influences between neighboring towns (geographic weights); lag importance connected to cyclicity and peak cases for extended COVID-19 case count and vaccine data; “remembering” high variance through cross validation; and including both the “sum” (total) of covid/vaccine cases to capture fluctuations throughout the pandemic, and the “average” cases to preserve moderate case count values.

Including town-level mobility measures can significantly improve the predictive power of models and explain an additional 75% of unexplained case variation. This is also the case if using only one town-level mobility measure and proper interaction terms in a panel-regression model. For example, holistic network measures can further boost the predictive power where behavioral responses are large, and mobility parameters can extend the forecasting window by a few weeks (up to 4). This is especially true during the first phase of the pandemic, diminishing later because of learning (people learning how to be mobile without putting themselves in risk) and changes in intervention policies, particularly the vaccinations.

With respect to town level NLP, there are two sets of implications, one practical, one substantive. Practically, an example embodiment provides a useful new tool for predicting COVID-19 cases at a level more fine-grained than counties, using an open and publicly available method that easily allows for social media data to be combined with the myriad other data sources used for prediction. More fundamentally, an example embodiment employes a bottom-up approach that uses a full array of words and phrases and can be applied to any predictive task: diseases, public opinion, voting, etc. Substantively, an example embodiment of a new Bayesian model shows that, however data is subdivided or aggregated, there are essentially just two modes of Covid-predictive speech: first, most speech related to COVID-19 is positively correlated with cases, particularly when aggregated. Second, talk about vaccination, which is either unrelated or positively related to COVID-19 early in the pandemic, becomes strongly negatively related to COVID-19 in the second year, when vaccinations were a practical concern. Further, it was found that emotionally, all talk predictive of COVID-19 is negatively toned, even vaccine-related talk, and that joyous talk is not predictive of cases.

An example embodiment may employ data stored in a relational database that also automatically retrieves and loads COVID-19 cases and vaccinations, and may automate the Twitter data collection pipeline. Such data may be called via an example embodiment of a Web App, which may insert OLS predictions and, for non-limiting example, the top 10 Twitter words/phrases determined to infer an uptick in COVID-19, or a downtick in COVID-19, per town, per week. Practically, such Web App may provide an interface where intelligence community, emergency, public health, and other relevant authorities can access accurate insights into potential disease outbreaks at a local level and evaluate supply chain stresses for local hospitals, etc. The interface does not undermine public trust owing to protection of Personally Identifiable Information (PII). No sensitive data is exposed to misappropriation.

As disclosed above, disease prediction is useful, but existing predictive models have often performed poorly, particularly during times of rapid change. The appeal of using social media for disease prediction is that, unlike many other data sources, it is highly dense in time and space, and spans almost any conceivable topic of human endeavor. However, despite many previous efforts, harnessing this immense data source to do real-world prediction, and provide insights into the opinions and behaviors that underly these predictions, remains elusive. An example embodiment disclosed herein may augment existing data streams for predicting COVID-19, generate new insights into COVID-19-related opinion, and lay the groundwork for a more general-purpose tool for predicting any manner of social phenomena.

An example embodiment disclosed herein may predict COVID-19 using social media text. The following tasks may enable same: 1) create a social media dataset of sufficient temporal density and geographical granularity to be useful for localized prediction. This is challenging because most social media data is restricted in various ways, most significantly in identifying geolocation; 2) develop machine learning methods for utilizing vast quantities of social media text in order to predict COVID-19 cases at the local level, and rigorously validate that those text-augmented predictions truly are more accurate than text-free baseline models; and, 3) develop new statistical models for interpreting the text selected by the predictive methods to generate new insights into COVID-19-associated behaviors and speech.

To achieve task 1), above, a new, open, public-API technique for geolocating Twitter data at a scale an order of magnitude denser than existing public methods was developed. These methods were developed and applied at the town level but generalizable to subnational regions worldwide. For task 2), above, existing machine learning methods were built upon in order to construct a model that takes in tens of thousands of words and phrases at the town-week level and predicts COVID-19 cases one week in the future. Such model was tested rigorously against a robust text-free baseline model and results obtained show that the text approach improves upon the baseline, particularly during times of rapid COVID-19 change, and is roughly as accurate four weeks in advance as the baseline model is one week in advance. For task 3), above, a new Bayesian model was developed that, in combination with pre-existing Bayesian topic models, allows terms to be clustered terms and to identify not just how to predict COVID-19 cases, but track how those effects change over time across different waves and pandemic periods.

Practically, a useful new tool was produced for predicting COVID-19 cases at a level more fine-grained than counties, using an open and publicly available method that easily allows for social media data to be combined with the myriad other data sources used for prediction. More fundamentally, a bottom-up approach was employed using a full array of words and phrases that can be applied to any predictive task: diseases, public opinion, voting, etc. for non-limiting examples.

Substantively, an example embodiment of a new Bayesian model showed that, however data is subdivided or aggregated, there are essentially just two modes of COVID-19-predictive speech: first, most speech related to COVID-19 is positively correlated with cases, particularly when aggregated. But second, talk about vaccination, which was either unrelated or positively related to COVID-19 early in the pandemic (when it was just another topic of conversation), becomes strongly negatively related to COVID-19 in the second year, when vaccinations were a practical concern. Further, as disclosed above, emotionally, all talk predictive of COVID-19 was found to be negative, even vaccine-related talk; whereas joyous talk, in as much as it exists, is not predictive of cases.

While both the machine learning and Bayesian modelling may entail computationally intensive components, an example embodiment may employ parallelization to resolve same. An example embodiment of an approach to predictive analytics disclosed herein offers a number of innovations and improvements on previous methods. First, an example embodiment may employ a new, open, public-API approach to geolocating tweets which produces much denser data streams than previous methods, with perhaps as many as 100× as many geolocated tweets per location per week. Second, an example embodiment may employ the full set of available ngrams (words and phrases) that were used in any tweet to predict cases, not just a small set of keywords. And third, an example embodiment may employ a novel Bayesian model constructed to discover and explain what groups of semantically and emotionally related words and phrases are most predictive of COVID-19, and how these effects change over time.

A fundamental challenge in employing social media data for predictive analytics is obtaining data of sufficient geographical specificity. Many social media sources, such as Twitter, are highly temporally specific, often down to the second, but due to a combination of privacy concerns and the increased monetary value of geographical location information, social media companies are often less willing or able to part with geographically specific metadata. Indeed, Twitter famously removed much of its geolocation information a few years ago, largely for privacy protection reasons.

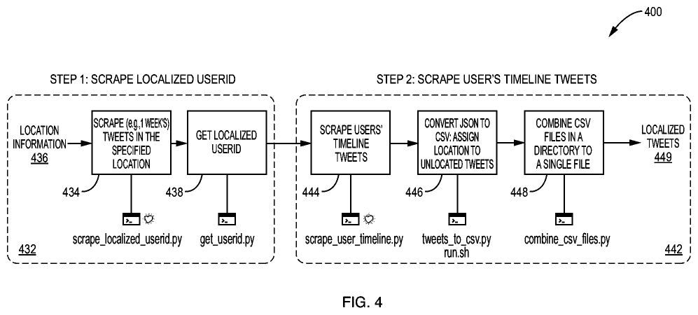

According to an example embodiment disclosed herein, instead of geolocating tweets using mixtures of metadata (such as latitude/longitude or Twitter's “location” field) and natural language processing methods (such as mentions of a location in the text of the tweet), the example embodiment instead locates individual accounts at the town level, and attributes all posts to their location, while also remaining cognizant of the fact that individuals often shift location over time. Assuming each individual has been correctly located, this approach vastly increases the dataset. For instance, rather than collecting only the tiny fraction of tweets that mention certain keywords and then only utilizing the tiny subset of those that can be confidently geolocated, an example embodiment disclosed herein can, instead, employ hundreds or thousands of times as many located tweets, albeit covering topics that potentially range far beyond COVID-19. An example embodiment of such a method is disclosed below with regard to FIG. 4.

FIG. 4 is a flow diagram 400 of an example embodiment of a method for large-scale geolocation of non-geolocated tweets. As shown in the flow diagram 400, an iterative approach is taken where, in a first phase 432 tweets are scraped 434 based on location information 436 obtained via Twitter's rather sparse remaining geolocation functions. A localized userId is determined 438. For example, associated meta-information about the authors of each of those tweets is gathered and if the author (user) appears to be located in a town of a particular state (based on either Twitter's “location” field or a user-supplied location field), the method continues to a second phase 442 and a scrape 444 of their entire available timeline is performed. At that point, the method may perform file format conversion 446, attribute each tweet to the author's location based on the tweet metadata and, if that is missing (as is generally the case), an example embodiment may impute the tweet's location based on inferences about the author's hometown. At no point does the method seek to localize a user or tweet to a location more specific than a town and, thus, does not undo the privacy protections Twitter put in place in largely removing specific latitude and longitude geolocation information. The method may combine 448 files in directory to a single file that may be output to provide localized tweets 449. Such localized tweets may be employed by a model disclosed herein that may be configured to model behavioral activity and thoughts.

The result of this process in a state, such as Massachusetts for non-limiting example, is that approximately 30,000 unique users were located at the town level, with over 30 million individual tweets between January 2020 and March 2022. This averages to over 700 tweets for each of the 350 towns per week, although the distribution is highly skewed towards more populous towns.

To validate such geolocation method, a manual check was performed for a subset of users, ascertaining that their assigned locations plausibly matched the content in their tweets. The manual overview also found that few of the users appeared to be bots, although some were businesses with potential numerous individuals contributing to the Twitter stream. Such is plausible because few bots tend to have their user locations set at a level of a modest-sized town in Massachusetts. It is assumed that many individuals and tweets may be incorrectly located, but that aggregate measures—e.g., word counts per town per week—will be relatively unbiased.

In order to predict COVID-19 cases using tweet text, an example embodiment of a model may reduce the corpus of localized tweets to n-gram counts at the town-week level, in which 1-grams (words) through 3-grams (3-word phrases) are counted. For computational reasons, year data for 2020 and 2021 were aggregated separately, retaining for each year the top 10,000 most common 1-, 2- and 3-grams, for a total of 30,000 ngrams. When combining the years, only ngrams that appear in both 2020 and 2021 were retained, leaving a set of approximately 20,000 ngrams that span 95 weeks from April 2020 to February 2022. In addition, to reduce the sparsity of the count matrix, any ngrams were removed that didn't appear at least 500 times in at least 100 different towns on at least 50 different weeks; while model performance did not seem to vary much with different settings, these were not heavily explored to prevent over-fitting. Finally, ngram counts were standardized by row (i.e., converted to percentages by town-week), and COVID-19 cases were converted to rates (i.e., divided by town population) and then logged.

A primary metric for predictive success is one-week-ahead prediction of (logged) COVID-19 case rates at the town-week level. This can be assessed by sweeping through the dataset, using any past data the model uses, and predicting cases for the subsequent week. These weekly predictions (for each of 172 towns) are then benchmarked against true case rates by taking the Mean Absolute Error (MAE) between prediction and truth at the town-week level. A fundamental question is whether adding the ngram data can improve upon the strongest baseline model that does not employ ngrams. For such purpose, a baseline was employed that does not use any additional information besides past cases (such as vaccination rates), but this baseline is nevertheless fairly robust, due to utilizing past cases, town level effects, and time trends. Averaging over all towns and weeks, it was found that the ngram model outperformed the baseline.

In addition to simply predicting COVID-19 cases, such an approach yields a wealth of terms that may provide insights into speech, opinions and behaviors associated with changes in COVID-19 cases. Rather than merely correlating term frequencies with COVID-19 cases, an example embodiment may focus on terms that are predictive of COVID-19 above and beyond what can be gleaned by overall case rates and region-specific variation. As a result, the terms discovered will be those that aren't merely correlates of overall COVID-19 rates (such as “COVID-19” itself) or correlates of regional demographic differences that are themselves correlated with COVID-19 (such as average wealth of a town). Rather, having absorbed these temporal and geographic differences into the fixed effects, an example embodiment disclosed herein instead finds terms that are predictive of differences in COVID-19 at the town-week level: regions that are anomalously higher or lower than expected based only on the town and week fixed effects.

One way to gain insight from these terms is simply to examine which terms are retained by the LASSO method for each week in a rolling prediction model. The T statistic threshold filtration approach reduces the total number of terms from about 15,000 to about 3,000, so the LASSO is working with these 3,000 terms for each weekly run. However, of these, over 1,000 are retained by the LASSO method in at least one week, with a very diffuse distribution: while the median number of terms retained for each week is around 40, most terms only appeal in two or fewer weeks, and only 11 terms are retained in more than 10 weekly runs. Of these, only one is COVID-19-related (“coronavirus case”), though a few other COVID-19-related terms appear in the top 100 (e.g., “health,” “crisis,” “positive_COVID-19_test,” and “COVID-19_case”). Most terms appear unrelated to COVID-19, and instead may reflect demographic differences that themselves are predictive of COVID-19: for instance, the top 10 terms include, “lmao,” “wanna,” “trynna,” and “bro.” Since these terms are those found when controlling for regional effects that might be associated with fixed demographic differences, this suggests that increases in these terms—which are generally used by younger and non-White users—may reflect increased online activity by these groups that in turn is predictive of rising cases. Speculatively, it is hypothesized that younger and non-White users may spend more time on Twitter when cases begin to rise (due to either shutdowns or voluntary withdrawal from other activities), which in turn may function as an early indicator of further increases in cases.

LASSO, however, is fundamentally designed to maximize out-of-sample prediction, not explanation. In addition to producing too many terms for easy interpretation, each weekly run introduces a significant amount of noise by reducing the sample size from our full dataset of 95 weeks to 4. Therefore, to get a less noisy measure of the terms that have the best overall association with COVID-19, a pooled approach may be employed, which is “cheating” for prediction purposes, but may yield the best overall insight into terms predictive of COVID-19 when controlling for time and location. However, because the dataset used spans nearly two years, five or six waves of COVID-19, numerous national crises, and significant shifts in attitudes towards COVID-19, it is believed that the association between terms and COVID-19 cases may vary significantly over this period: the words and phrases predictive of cases early on, during the initial crisis, may be very different than during the later phases where “normalcy” begins to return.

To strike a balance between the weekly models, which are attuned to changes in effects but are generally too noisy, and the pooled approach, which utilizes all observations but does not allow for changing effects, a Bayesian model was constructed that is designed to generate smoothly time-varying coefficients. The core structure of the Bayesian model is like the LASSO approach: the main difference is that the lagged case effects βt and (lagged) ngram effects βt are allowed to varying by week (hence the t subscript), and an example embodiment imposes priors to smooth out the autocorrelation among those changing effects. The core model is therefore:

γt,k˜wt+sk+βtyt—Lk+Xt−1,kβt

Additional Bayesian hierarchical structures include (1) βt N(βt−1, σ), i.e., autocorrelation among the lag case effects over time; and (2) βt N(βt−1, σ), i.e., autocorrelation among each ngram effect over time. An example embodiment also imposes (3) sk˜N(s, σ) (random region effects are normally distributed) and (4) wt˜N(wt−1, σ) (autocorrelation among weekly effects), as well as various hyperpriors.

Rather than applying this model to all 20,000 terms separately or jointly, which would be both computationally challenging and/or lead to massive collinearity among similar terms, three different ways were examined of first extracting and aggregating potentially salient terms: 1) Bivariate selection: those terms that emerge from the bivariate screening process described above, i.e., the terms most predictive of COVID-19 in the pooled model; 2) Keyword selection: a set of COVID-19-related keywords that are generated through a mixture of expert guidance and machine learning; and 3) emotion selection: a large corpus of sentiment words associated with six different emotions.

Since each of these approaches still produced far too many terms to be individually interpretable, terms were aggregated. From the first two selection methods, terms were aggregated into “topics” using a Latent Dirichlet Allocation (LDA) topic model. This finds clusters of correlated ngrams, which are then used to create composite variables that are aggregates of the ngrams in each topic. For the emotion terms, the ngrams assigned to each emotion were aggregated.

Notably, such approaches (1 and 2) were found to produce very similar results despite very different methods. For selection method 1, a specific procedure is to first select only terms with an absolute T statistic above 2 and frequency percentile>0.7 from the bivariate regressions, and apply a topic model with 5 topics, having found that more topics yielded increasing redundancy (i.e., topics with very similar ngrams). Of these five topics, one was clearly COVID-19-related (top words: “COVID-19, coronavirus, president”), one was related the president and elections (“president, count, win”) and three were unrelated and primarily seemed to be picking up seasonal or short-term events (e.g., Christmas, the Bruins, and a member of Congress in the news). Five aggregate variables were created, one for each topic, by summing the counts of the top 15 ngrams for each topic in each town-week.

When including these aggregate topic variables in the Bayesian model (one at a time), it was found that each of them had similar predictive effects over time. It was found that the effects for each week of the COVID-19-related topic, which is largely positive (i.e., positively correlated with COVID-19 cases) for most of the first year of the pandemic, then less significant for the second year. Upon closer inspection, it was realized that there are many more ngrams that are positively related to COVID-19 than negative, so the thresholding approach had selected mainly terms that were positively related and had similar relations to COVID-19 in aggregate, explaining why all five topics tended to have similar trajectories. To better examine potentially negatively predictive terms, two additional aggregate variables were created: the aggregate of all ngrams with T statistic>2, and the aggregate of all ngrams with T statistic<−1. Estimating those two aggregate variables in the Bayesian model yielded a positive aggregate that looked much like the “covid” topic and indeed shared many of the same words, while the negative aggregate began positively correlated with COVID-19, but by the end of the pandemic was strongly negatively correlated with cases. The keyword approach elucidates why.

For the keyword selection (method 2), in order to focus specifically on COVID-19-related terms a set of COVID-19-related ngrams was constructed using a three step procedure: first a small list of terms drawn from literature; then this list was augmented using a deep learning word embedding trained our or dataset to find semantically similar terms; and finally all bigrams and trigrams were selected that included any of the unigrams from step two. This procedure yielded a subset of about 200 ngrams out of our total 20K corpus. A 5-topic model was then tested, but its found that even with 5 topics, there was a high level of redundancy across topics, and in fact this persisted until the number of topics was reduced to 2. With two topics, there were two very distinctive groups: the first contained all manner of COVID-19-related ngrams: “coronavirus, quarantine, test, COVID-19, test positive, positive_COVID-19, test_positive_COVID-19, death, testing, corona, pandemic, hospital” etc. The second contained almost exclusively vaccine-related ngrams “vaccine, vaccinate, vaccination, COVID-19 vaccine, vaccinated, get vaccinate, get vaccine, fully vaccinate, COVID-19 vaccination, get vaccinated, COVID-19 relief, get COVID-19 vaccine” etc. When creating aggregate variables out of these two keyword-based topics and including them in the Bayesian model, it was found that the first closely matches the positive term aggregate from selection method 1, while the vaccine-related topic closely matches the negative term aggregate from method 1. Indeed, the vaccine-related topic offers what appears to be a cleaner signal: it begins quite positive early in the pandemic but finishes quite negative. This suggests that most of the negatively correlated prediction found by our bivariate regression is due to vaccine talk: early in the pandemic, talking about a vaccine is just a general (positive) correlate with COVID-19 cases, as is most other COVID-19-related speech; but later in the pandemic, when vaccines became available, the relationship turns negative: more talk about vaccines predicts less COVID-19, perhaps because it is associated with minutely increased vaccine uptake.

Finally, having seen the topics of discussion that are predictive of COVID-19, it was also of interest to know how and with what emotions these topics are being discussed. A common theory has been that rising or falling levels of anxiety or fear affect individual levels of mobility and personal protective behaviors, which in turn drives cases. Conversely, when discussing vaccination, it may be that these specific discussion may be celebratory. To test these theories, a corpus of emotion-related words were used to construct six emotional aggregates, each comprising a few thousand terms (many of which were not in our corpus) assigned by experts as associated with each emotion: anger, disgust, fear, joy, sadness, and surprise. When included in the Bayesian model, only two of these six emotional aggregates appeared significant: disgust and sadness, with sadness the most consistently predictive of COVID-19 of all the aggregate variables. Both emotions are also positively correlated with our COVID-19-related aggregates, but none of the emotions were correlated with the vaccine-related aggregate. Thus, while most speech that is positively predictive of COVID-19 is sad, the vaccine speech is neither happy nor sad overall.

It should be understood that an example embodiment of a system disclosed herein may be considered to be a tool infrastructure and may be applied to any real-world phenomena measured at sufficient geo-spatio-temporal scale. An example embodiment disclosed herein may employ a model that is generatlized to other situations, such as, virus severity/morbidity; a different virus/the flu; or other dynamic phenomena (disinvestment, crime, chem-bio warfare) etc., for non-limiting examples.

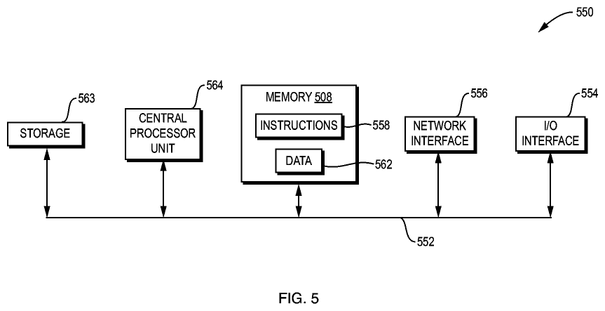

FIG. 5 is a block diagram of an example of the internal structure of a computer 550 in which various embodiments of the present disclosure may be implemented. The computer 550 contains a system bus 552, where a bus is a set of hardware lines used for data transfer among the components of a computer or digital processing system. The system bus 552 is essentially a shared conduit that connects different elements of a computer system (e.g., processor, disk storage, memory, input/output ports, network ports, etc.) that enables the transfer of information between the elements. Coupled to the system bus 552 is an I/O device interface 554 for connecting various input and output devices (e.g., keyboard, mouse, display monitors, printers, speakers, microphone, etc.) to the computer 550. The I/O device interface 554 may, for non-limiting example, be employed to provide an application programming interface (API) to interface with a ML model(s) disclosed herein. A network interface 556 allows the computer 550 to connect to various other devices attached to a network (e.g., global computer network, wide area network, local area network, etc.). The network interface 556 may be used to interface with a ML model(s) to perform validation, ranking, and weighting for non-limiting examples. Memory 558 provides volatile or non-volatile storage for computer software instructions 558 and data 562 that may be used to implement embodiments (e.g., method 300) of the present disclosure, where the volatile and non-volatile memories are examples of non-transitory media. The memory 558 may, for non-limiting example, be employed to store ML model(s) as well as training data. Disk storage 563 also provides non-volatile storage for the computer software instructions 558 and data 562 that may be used to implement embodiments (e.g., method 300) of the present disclosure. The disk storage 563 may be employed to store high dimensional data streams, disclosed above, in a relational database for non-limiting example. A central processor unit 564 is also coupled to the system bus 552 and provides for the execution of computer instructions. The central processor unit 564 may, for non-limiting example, perform pre-processing of data streams and feature selection disclosed above.

Further example embodiments disclosed herein may be configured using a computer program product; for example, controls may be programmed in software for implementing example embodiments. Further example embodiments may include a non-transitory computer-readable-medium that contains instructions that may be executed by a processor, and, when loaded and executed, cause the processor to complete methods and techniques described herein. It should be understood that elements of the block and flow diagrams may be implemented in software or hardware, such as via one or more arrangements of circuitry of FIG. 5, disclosed above, or equivalents thereof, firmware, a combination thereof, or other similar implementation determined in the future.

In addition, the elements of the block and flow diagrams described herein may be combined or divided in any manner in software, hardware, or firmware. If implemented in software, the software may be written in any language that can support the example embodiments disclosed herein. The software may be stored in any form of computer readable medium, such as random-access memory (RAM), read only memory (ROM), compact disk read-only memory (CD-ROM), and so forth. In operation, a general purpose or application-specific processor or processing core loads and executes software in a manner well understood in the art. It should be understood further that the block and flow diagrams may include more or fewer elements, be arranged or oriented differently, or be represented differently. It should be understood that implementation may dictate the block, flow, and/or network diagrams and the number of block and flow diagrams illustrating the execution of embodiments disclosed herein.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A computer-implemented method for predictive analytics, the computer-implemented method comprising:
combining, by a machine-learning (ML) predictor model, multiple independent data streams received from respective diverse ML models, the multiple independent data streams including respective features indicative of an observable event, the combining producing at least one combination of the respective features;
generating, by the ML predictor model, a prediction of the observable event, the prediction based on the at least one combination produced; and
outputting, by the ML predictor model, a representation of the prediction generated.

2. The computer-implemented method of claim 1, wherein the observable event includes at least one of: an outbreak of an infectious disease, release of a chemical or biological warfare agent, pattern of migration, or other observable event.

3. The computer-implemented method of claim 1, wherein the representation is a visual representation and wherein the outputting includes:
outputting the visual representation to an electronic display device; and
displaying the visual representation on the electronic display device.

4. The computer-implemented method of claim 1, wherein the combining includes ranking the respective features and weighting the respective features ranked.

5. The computer-implemented method of claim 1, further comprising producing the multiple independent data streams and wherein the producing includes:
performing, by at least one respective diverse ML model of the respective multiple diverse ML models, natural language processing (NLP) on input data, wherein the NLP includes extracting natural language information from the input data, and wherein at least a portion of the respective features includes the natural language information extracted.

6. The computer-implemented method of claim 1, further comprising customizing a respective diverse ML model of the respective diverse ML models to produce an independent data stream of the multiple independent data streams based on social media analytics, mobility data analytics, dynamic data analytics, or static data analytics.

7. The computer-implemented method of claim 1, further comprising customizing the respective multiple diverse ML models to produce the respective features by filtering input data based on a database of terms, wherein the terms include natural language, and wherein the terms are categorized into sentiments in the database.

8. The computer-implemented method of claim 1, further comprising producing the multiple independent data streams by the respective multiple diverse ML models, wherein the multiple independent data streams produced are associated with a common geographical area, common timeframe, and common user pool, and wherein at least a portion of users of the common user pool are determined to be located within the common geographical area for at least a portion of the common timeframe.

9. The computer-implemented method of claim 1, further comprising producing the multiple independent data streams, by the multiple diverse ML models, with a consistent format, and wherein the consistent format is associated with NLP data.

10. The computer-implemented method of claim 1, wherein generating the prediction includes employing at least one of: statistical modeling, Bayesian modeling, least absolute shrinkage, and selection operator (Lasso) regression modeling, or topic modeling.

11. A system for predictive analytics, the system comprising:
a machine learning (ML) predictor model; and
diverse ML models coupled to the ML predictor model, the ML predictor configured to:
combine multiple independent data streams received from respective diverse ML models of the diverse ML models, the multiple independent data streams including respective features indicative of an observable event, the combining producing at least one combination of the respective features;
generate a prediction of the observable event, the prediction based on the at least one combination produced; and
output a representation of the prediction generated.

12. The system of claim 11, wherein the observable event includes at least one of: an outbreak of an infectious disease, release of a chemical or biological warfare agent, pattern of migration, or other observable event.

13. The system of claim 11, wherein the system is a computer-based system, wherein computer-based system includes an electronic display device, wherein the representation is a visual representation, wherein the ML predictor model is further configured to output the visual representation to the electronic display device, and wherein the electronic display device is configured to display the visual representation.

14. The system of claim 11, wherein the ML predictor model is further configured to rank the respective features and weight the respective features ranked.

15. The system of claim 11, wherein at least one respective diverse ML model of the respective multiple diverse ML models is configured to produce an independent data stream of the multiple independent data streams by performing natural language processing (NLP) on input data, wherein the NLP includes extracting natural language information from the input data, and wherein at least a portion of the respective features includes the natural language information extracted.

16. The system of claim 11, wherein a respective diverse ML model of the respective diverse ML models is customized to produce an independent data stream of the multiple independent data streams based on social media analytics, mobility data analytics, dynamic data analytics, or static data analytics.

17. The system of claim 11, wherein the respective multiple diverse ML models are customized to produce the respective features by filtering input data based on a database of terms, wherein the terms include natural language, and wherein the terms are categorized into sentiments in the database.

18. The system of claim 11, wherein the multiple independent data streams are associated with a common geographical area, common timeframe, and common user pool, and wherein at least a portion of users of the common user pool are determined to be located within the common geographical area for at least a portion of the common timeframe.

19. The system of claim 11, wherein the multiple independent data streams have a consistent format, wherein the consistent format is associated with NLP data, and wherein, to generate the prediction, the ML predictor model is further configured to employ at least one of: statistical modeling, Bayesian modeling, least absolute shrinkage, and selection operator (Lasso) regression modeling, or topic modeling.

20. A non-transitory computer-readable medium for predictive analytics, the non-transitory computer-readable medium having encoded thereon a sequence of instructions which, when loaded and executed by at least one processor, causes the at least one processor to:
combine multiple independent data streams received from respective diverse machine learning (ML) models, the multiple independent data streams including respective features indicative of an observable event, the combining producing at least one combination of the respective features;
generate a prediction of the observable event, the prediction based on the at least one combination produced; and
output a representation of the prediction generated.
